SEQUENCE LISTING (24 sequences)

SEQ ID NO: 1 - protein, 6 aa, Artificial Sequence
  Peptide tight junction agonist
FCIGRL

SEQ ID NO: 2 - protein, 6 aa, Artificial Sequence
  Peptide tight junction agonist
XCIGRL

SEQ ID NO: 3 - protein, 6 aa, Artificial Sequence
  Peptide tight junction agonist
FXIGRL

SEQ ID NO: 4 - protein, 6 aa, Artificial Sequence
  Peptide tight junction agonist
FCXGRL

SEQ ID NO: 5 - protein, 6 aa, Artificial Sequence
  Peptide tight junction agonist
FCIXRL

SEQ ID NO: 6 - protein, 6 aa, Artificial Sequence
  Peptide tight junction agonist
FCIGXL

SEQ ID NO: 7 - protein, 6 aa, Artificial Sequence
  Peptide tight junction agonist
FCIGRX

SEQ ID NO: 8 - protein, 6 aa, Artificial Sequence
  Peptide tight junction agonist
XXIGRL

SEQ ID NO: 9 - protein, 6 aa, Artificial Sequence
  Peptide tight junction agonist
XCXGRL

SEQ ID NO: 10 - protein, 6 aa, Artificial Sequence
  Peptide tight junction agonist
XCIXRL

SEQ ID NO: 11 - protein, 6 aa, Artificial Sequence
  Peptide tight junction agonist
XCIGXL

SEQ ID NO: 12 - protein, 6 aa, Artificial Sequence
  Peptide tight junction agonist
XCIGRX

SEQ ID NO: 13 - protein, 6 aa, Artificial Sequence
  Peptide tight junction agonist
FXXGRL

SEQ ID NO: 14 - protein, 6 aa, Artificial Sequence
  Peptide tight junction agonist
FXIXRL

SEQ ID NO: 15 - protein, 6 aa, Artificial Sequence
  Peptide tight junction agonist
FXIGXL

SEQ ID NO: 16 - protein, 6 aa, Artificial Sequence
  Peptide tight junction agonist
FXIGRX

SEQ ID NO: 17 - protein, 6 aa, Artificial Sequence
  Peptide tight junction agonist
FCXXRL

SEQ ID NO: 18 - protein, 6 aa, Artificial Sequence
  Peptide tight junction agonist
FCXGXL

SEQ ID NO: 19 - protein, 6 aa, Artificial Sequence
  Peptide tight junction agonist
FCXGRX

SEQ ID NO: 20 - protein, 6 aa, Artificial Sequence
  Peptide tight junction agonist
FCIXXL

SEQ ID NO: 21 - protein, 6 aa, Artificial Sequence
  Peptide tight junction agonist
FCIXRX

SEQ ID NO: 22 - protein, 6 aa, Artificial Sequence
  Peptide tight junction agonist
FCIGXX

SEQ ID NO: 23 - protein, 6 aa, Artificial Sequence
  Peptide tight junction agonist
SLIGRL

SEQ ID NO: 24 - protein, 399 aa, Vibrio cholerae
MSIFIHHGAPGSYKTSGALWLRLLPAIKSGRHIITNVRGLNLERMAKYLKMDVSDISIEFIDTDHPDGRLTMARFWHWARKDAFLFIDECGRIWPPRLTATNLKALDTPPDLVAEDRPESFEVAFDMHRHHGWDICLTTPNIAKVHNMIREAAEIGYRHFNRATVGLGAKFTLTTHDAANSGQMDSHALTRQVKKIPSPIFKMYASTTTGKARDTMAGTALWKDRKILFLFGMVFLMFSYSFYGLHDNPIFTGGNDATIESEQSEPQSKATAGNAVGSKAVAPASFGFCIGRLCVQDGFVTVGDERYRLVDNLDIPYRGLWATGHHIYKDKLTVFFETESGSVPTELFASSYRYKVLPLPDFNHFVVFDTFAAQALWVEVKRGLPIKTENDKKGLNSIF

(19) United States
(12) Patent Application Publication
(10) Pub. No.: US 2009/0252672 A1
(43) Pub. Date: Oct. 8, 2009

(54) NASAL DELIVERY OF THERAPEUTIC AGENTS USING TIGHT JUNCTION AGONISTS

(76) Inventors: Natalie D. Eddington, Owings Mills, MD (US); Keon-Hyoung Song, Daejeon (KR); Zeynep Teksin, Yukaryayrancy (TR); Alan A. Cross, Chevy Chase, MD (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/300,392

(22) PCT Filed: May 11, 2007
(86) PCT No.: PCT/US07/68792

§ 371 Date: Mar. 16, 2009

Related U.S. Application Data
(60) Provisional application No. 60/799,336, filed on May 11, 2006.

(51) Int. Cl. A61K 47/42; A61K 38/02; A61K 38/13; A61K 31/7028; A61K 38/28; A61K 51/00; A61K 38/43; A61K 31/70; A61P 3/10

(52) U.S. Cl. 424/1.11; 514/773; 514/11; 514/50; 514/12; 514/4; 424/158.1; 424/94.1; 514/23

(57) ABSTRACT

The invention relates to a therapeutic composition comprising a therapeutically effective amount of one or more therapeutic agents and a nasal mucosa absorption-enhancing amount of one or more tight junction agonists. The invention further relates to a method of treating a subject comprising intranasally administering the composition of the invention to the subject.

### CROSS REFERENCE TO RELATED APPLICATIONS

This application claims priority to U.S. provisional patent application Ser. No. 60/799,336, filed May 11, 2006, the contents of which are specifically incorporated herein by reference.

### STATEMENT REGARDING FEDERALLY SPONSORED RESEARCH

This research was funded by NIH Grants 2-R01 EB02771 and MH067507. The United States Government has certain rights in the invention.

### BACKGROUND OF THE INVENTION

The low bioavailability (BA) of efficacious pharmacotherapeutic drugs continues to be a major obstacle in drug development and in many instances may be the deciding factor on whether or not a potent agent is developed. These therapeutic agents experience low BA after mucosal administration due to poor absorption or susceptibility to first pass metabolism. The importance of the search for an efficient novel drug delivery system to overcome this problem cannot be overemphasized. In addition, development of effective means of delivery of a drug to a mucosal membrane that does not suffer the extreme environment of the gut would be an advantage. A means of enhancing the absorption of these drugs by the nasal membrane would significantly extend their therapeutic usefulness as well as decreasing the dose required to produce efficacy.

Absorption enhancers, including surfactants, fatty acids, and chitosan derivatives, have been used to modify bioavailability by either disruption of the cell membrane or modulation of the tight junctions (TJ) (1). In general, the optimal absorption enhancer should possess the following qualities: its effect should be reversible, it should provide a rapid permeation enhancing effect on the cellular membrane of the mucosa, and it should be non-cytotoxic at the effective concentration level and without deleterious and/or irreversible effects on the cellular membrane or cytoskeleton of the TJ. Zonula Occludens Toxin (ZOT), a 44.8 kDa protein (399 amino acids; AA) located in the cell envelope of the bacterial strain Vibrio cholerae, is capable of reversibly opening the TJ between cells and increasing the paracellular transport of many drugs in a non-toxic manner (2-7). ZOT binds to a specific receptor on the luminal surface of the intestine and reversibly opens the TJ between intestinal epithelial cells (2-7). Intensive investigation of the biological activity of ZOT as an absorption enhancer was triggered by reports of effective oral administration of insulin with ZOT in diabetic rats (4). Recently, a smaller 12 kDa fragment (AA 265-399) of ZOT, referred to as delta G (ΔG), was introduced as the biologically active fragment of ZOT (8). ΔG, a biologically active 12 kDa fragment of ZOT, was isolated and displayed the intrinsic activity of reversibly modulating TJ thus increasing the paracellular transport of drugs (8). The effect of the zonulin/zot receptor may also be related to that of an entirely different receptor, the protease activated receptor-2 (PAR-2) receptor. PAR-2 agonists are peptides having 6 amino acid residues, with 4 of the amino acids being identical to that of the ZOT/Zonulin receptor binding motif (XX-IGRL) (8). Intracolonic infusion of a 5 μg dose of the PAR-2 agonist, SLIGRL, resulted in a 2-fold increase in the paracellular permeability of [⁵¹Cr]-EDTA (9). ZOT and ΔG trigger a cascade of intracellular events mediated by protein kinase C with polymerization of soluble G-actin, subsequent displacement of proteins from the junctional complex, and loosening of TJ (3). Thus, they can reversibly open the intestinal TJ in a non toxic manner (2-7, 10).

Studies in our laboratory have shown that ZOT enhances the intestinal transport of drug candidates of varying molecular weight (mannitol, PEG4000, Inulin, and sucrose) or low BA (paclitaxel, acyclovir, cyclosporin A, and doxorubicin) across Caco-2 cell monolayers (6, 7). Moreover, the transport enhancing effect of ZOT is reversible and no toxicity is observed (2, 7). In addition, ΔG significantly increased the in vitro transport of paracellular markers (mannitol, PEG4000, and Inulin) in a nontoxic manner and the in vivo absorption of low BA therapeutic agents (cyclosporin A, ritonavir, saquinavir, and acyclovir) (11-12).

In previous studies with ZOT, bioavailability of oral insulin coadministered with ZOT (4.4×10⁻¹⁰ mol/kg) was sufficient to lower serum glucose concentrations to levels comparable to those obtained after parenteral injection of the hormone in diabetic rats (4). ZOT (0.45×10⁻¹⁰ mol/ml, 0.89×10⁻¹⁰ mol/ml) increased the permeability of molecular weight markers (sucrose, Inulin) over a range of 130% to 195% and chemotherapeutic agents (paclitaxel, doxorubicin) across the bovine brain microvessel endothelial cells (BBMEC) (13). In addition, ZOT (0.22 to 0.89×10⁻¹⁰ mol/ml) enhanced the transport of agents of varying molecular weights (mannitol, PEG4000, Inulin) or low bioavailability (doxorubicin, paclitaxel, acyclovir, cyclosporin A, anticonvulsant enaminones) up to 30 fold across Caco-2 cell monolayers, without modulating the transcellular transport (6, 7).

Also, studies have shown that ΔG (0.83 to 1.50×10⁻⁸ mol/ml) increased the transport of paracellular markers (mannitol, Inulin, PEG4000) by 1.2 to 2.8-fold across Caco-2 cells relative to the transepithelial transport of markers in its absence (10, 11). After intradermal (ID) administration to rats, ΔG (3.48 to 6.00×10⁻⁸ mol/kg) displayed high intrinsic biological activity with paracellular markers (mannitol, Inulin, PEG4000) and some low bioavailable drugs (CsA, ritonavir, saquinavir, acyclovir) (10-12). Moreover, in vivo administration of ΔG effected up to 57 and 50-fold increases in Cₘₐₓ and AUC, respectively, of CsA after metabolic protection was provided (12). Protease inhibitors (e.g., a mixture of bestatin, captopril, and leupeptin) are needed to minimize enzymatic degradation of ΔG by proteases or peptidases in the gut (11, 12).

In vivo use of either ZOT or ΔG has several disadvantages, however. The isolation and purification protocols are tedious and time consuming. Moreover, the yield of protein is not sufficient for extensive use. Amino acid comparison between ZOT active fragment and Zonulin, combined with site-directed mutagenesis experiments, confirmed the presence of a hexapeptide receptor-binding domain toward the amino terminus of the processed ZOT.

The active peptide FCIGRL was synthesized and found to retain the permeating effect on intercellular TJ which characterizes ZOT and ΔG. The active peptide is now termed AT1002.

### SUMMARY OF THE INVENTION

The method and composition of the invention relates broadly to methods and compositions for enhancing absorption of a therapeutic agent by mucosal tissues. Thus, the composition can be administered to a subject by any suitable route, including intranasally. In one aspect, the composition is directly or indirectly administered to the nasal mucosa. For example, the methods and compositions of the invention are useful for enhancing absorption in the nasal turbinates, sinuses and associated structures.

In one aspect, the invention comprises a therapeutic composition comprising a therapeutically effective amount of one or more therapeutic agents and a nasal mucosa absorption enhancing amount of one or more tight junction agonists. As used herein, a “tight junction agonist” is a compound that mediates or facilitates or augments the physiological, transient opening of tight junctions. Tight junctions are structures that form a barrier between adjacent epithelial cells (Johnson and Quay, Expert Opin. Drug Deliv. 2005 March; 2(2):281-98). An example of a tight junction agonist is zonula occludens toxin (ZOT), which is produced by Vibrio cholerae. A ZOT receptor agonist is a tight junction agonist which is believed to mediate tight junction opening through the same receptor utilized by ZOT.

In another aspect, the invention comprises a composition wherein at least one therapeutic agent is selected from the group consisting of an antibiotic, an anti-inflammatory, an analgesic, an immunosuppressant, and a peptide hormone.

The compositions of the invention can comprise a peptide hormone which can be insulin.

The composition of the invention can also comprise one or more therapeutic agents wherein at least one of the one or more therapeutic agents is selected from the group consisting of a small molecule, a peptide, a protein, a protease inhibitor, a lipid, a carbohydrate, and combinations thereof.

In one aspect, the composition is in aqueous solution. In another particular aspect, the composition is in a solid state. The solid state composition can be a powder, for example, a microcrystalline powder or an amorphous powder.

The composition can further comprise one or more pharmaceutically acceptable excipients.

In still another aspect, the invention comprises a composition wherein at least one of the one or more tight junction agonists is a peptide comprising the sequence FCIGRL and the composition further comprises at least one protease inhibitor and one or more therapeutic agents selected from the group consisting of a small molecule, a peptide, a protein, a protease inhibitor, a lipid, and a carbohydrate, and combinations thereof.

In another aspect, the invention comprises a method of treating a subject comprising intranasally administering to the subject the composition of the invention. In a particular aspect, the composition can comprise one or more therapeutic agents and an intestinal absorption enhancing amount of one or more tight junction agonists. The subject can be a mammal. In one particular aspect, the subject is a human.

In yet another aspect, the invention comprises a method of treating diabetes in an animal in need thereof, comprising: intranasally administering to the animal a composition comprising an insulin, a derivative of an insulin, or a combination thereof, and a nasal mucosa absorption enhancing amount of one or more tight junction agonists.

### BRIEF DESCRIPTION OF THE FIGURES

FIG. 1 shows the amino acid sequence of ZOT. The residues in bold text (265-399) are ΔG, the biologically active fragment of ZOT, and the boxed sequence (288-293) is AT1002, the active domain of ZOT.

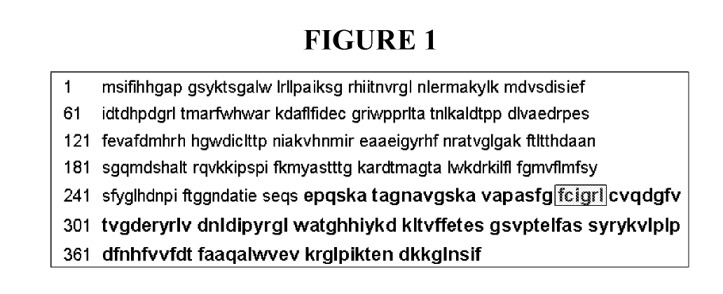

FIG. 2 shows the uptake of [³H]-AZT into the blood plasma of FVB mice, showing control (light gray) and co-administration with FCIGRL at 2 mg/kg (dark gray).

FIG. 3 shows the uptake of [³H]-Saquinavir into the blood plasma of FVB mice, showing control (light gray) and co-administration with FCIGRL at 2 mg/kg (dark gray).

FIG. 4 shows the average plasma concentration versus time profile for [¹⁴C] PEG4000 in jugular cannulated Sprague-Dawley rats following the intranasal administration of treatments. i.e., PEG4000 (), PEG4000/AT1002(5 mg/kg) (◯), and PEG4000/AT1002(10 mg/kg) (▾). Each data point represents the mean±SEM of 4-5 rats. * Significant at p<0.05 compared to PEG4000 only of each same time point.

FIG. 5 shows the Average plasma concentration versus time profile for [¹⁴C] inulin in jugular cannulated Sprague-Dawley rats following the intranasal administration of treatments. i.e., inulin (), inulin/AT1002(5 mg/kg) (◯), and inulin/AT1002(10 mg/kg) (▾). Each data point represents the mean±SEM of 4-5 rats. * Significant at p<0.05 compared to inulin only of each same time point.

FIG. 6 shows average plasma concentration versus time profile for sCT (15 mg/kg) in jugular cannulated Sprague-Dawley rats following the intra-nasal administration of control and three treatments. i.e., sCT only (), sCT/AT10022 (∇), 5 (▪) or 10 mg/kg (⋄). Each data point represents the mean±SEM of 3-5 rats. * Significant at p<0.05 compared to sCT only (control).

FIG. 7 shows average plasma concentration at each Tmax versus concentration profile for sCT (15 mg/kg) in jugular cannulated Sprague-Dawley rats following the intra-nasal administration of control and three treatments. Tmax were 30 min for sCT only and 20 min for sCT with AT1002 formulations. Each data point represents the mean±SEM of 3-5 rats. * Significant at p<0.05 compared to sCT only (control).

### DETAILED DESCRIPTION

Zonula Occludens Toxin (ZOT) and its biologically active fragment, ΔG, have been shown to reversibly open TJ in endothelial and epithelial cells. Recently, a six-residue synthetic peptide, H-FCIGRL-OH (AT1002), was identified that retains the permeating effect on intercellular TJ characteristic of ZOT. An object of this invention is to demonstrate the biological activity of AT1002 on enhancing uptake of several agents, including AZT, after intranasal administration.

Tight Junction Agonists

Compositions of the invention typically comprise one or more tight junction agonists. A tight junction agonist facilitates absorption of a therapeutic agent. Thus, a tight junction agonist as used herein is a compound that mediates the physiological, transient opening of tight junctions. In some embodiments, a tight junction agonist may operate by binding to the ZOT receptor, i.e., may be a ZOT receptor agonist.

In some embodiments, a tight junction agonist may comprise a peptide comprising the amino acid sequence FCIGRL and/or functional derivatives of this sequence. Functional derivatives of peptide FCIGRL include, for example, Xaa₁ Cys Ile Gly Arg Leu (SEQ ID NO: 2), Phe Xaa₂ Ile Gly Arg Leu (SEQ ID NO: 3), Phe Cys Xaa₃ Gly Arg Leu (SEQ ID NO: 4), Phe Cys Ile Xaa₄ Arg Leu (SEQ ID NO: 5), Phe Cys Ile Gly Xaa₅ Leu (SEQ ID NO: 6), and Phe Cys Ile Gly Arg Xaa₆ (SEQ ID NO: 7). Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met. In some embodiments, a tight junction agonist may consist of a peptide having the sequence FCIGRL and/or functional derivatives of this sequence as described herein.

Further, functional derivatives of peptide FCIGRL include: Xaa₁, Xaa₂ Ile Gly Arg Leu (SEQ ID NO: 8), Xaa₁ Cys Xaa₃ Gly Arg Leu (SEQ ID NO: 9), Xaa₁ Cys Ile Xaa₄ Arg Leu (SEQ ID NO: 10), Xaa₁ Cys Ile Gly Xaa₅ Leu (SEQ ID NO: 11), Xaa₁ Cys Ile Gly Arg Xaa₆ (SEQ ID NO: 12), Phe Xaa₂ Xaa₃ Gly Arg Leu (SEQ ID NO: 13), Phe Xaa₂ Ile Xaa₄ Arg Leu (SEQ ID NO: 14), Phe Xaa₂ Ile Gly Xaa₅ Leu (SEQ ID NO: 15), Phe Xaa₂ Ile Gly Arg Xaa₆ (SEQ ID NO: 16), Phe Cys Xaa₃ Xaa₄ Arg Leu (SEQ ID NO: 17), Phe Cys Xaa₃ Gly Xaa₅ Leu (SEQ ID NO: 18), Phe Cys Xaa₃ Gly Arg Xaa₆ (SEQ ID NO: 19), Phe Cys Ile Xaa₄ Xaa₅ Leu (SEQ ID NO: 20), Phe Cys Ile Xaa₄ Arg Xaa₆ (SEQ ID NO: 21), and Phe Cys Ile Gly Xaa₅ Xaa₆ (SEQ ID NO: 22). Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

When the tight junction agonist is a peptide, any length of peptide may be used. For example, an agonist may be about 3, about 4, about 5, about 6, about 7, about 8, about 9, about 10, about 11, about 12, about 13, about 14 or about 15 amino acids in length. In some embodiments, a peptide tight junction agonist may be from about 3 to about 12, from about 4 to about 12, from about 5 to about 12, from about 6 to about 12, from about 7 to about 12, from about 8 to about 12, from about 9 to about 12, from about 10 to about 12, from about 3 to about 10, from about 4 to about 10, from about 5 to about 10, from about 6 to about 10, from about 7 to about 10, from about 8 to about 10, from about 9 to about 10 amino acids in length. In some embodiments, a peptide tight junction agonist may be 9 amino acids or less in length. In some embodiments of the invention, peptides tight junction agonists do not encompass full length ZOT or zonulin.

Peptide agonists can be chemically synthesized and purified using well-known techniques, such as described in High Performance Liquid Chromatography of Peptides and Proteins: Separation Analysis and Conformation, Eds. Mant et al., C. R. C. Press (1991), and a peptide synthesizer, such as Symphony (Protein Technologies, Inc.); or by using recombinant DNA techniques, i.e., where the nucleotide sequence encoding the peptide is inserted in an appropriate expression vector, e.g., an E. coli or yeast expression vector, expressed in the respective host cell, and purified from the cells using well-known techniques.

Therapeutic Agents

Compositions of the invention typically comprise one or more therapeutic agents and/or immunogenic agents. Therapeutic agents that can be used in the compositions include agents that act on any organ of the body, such as heart, brain, intestine, or kidneys. Examples of suitable therapeutic agents include, but are not limited to, glucose metabolism agents (e.g., insulin), antibiotics, antineoplastics, antihypertensives, antiepileptics, central nervous system agents, and immune system suppressants.

The materials and methods of the invention may be used to enhance the uptake and bioavailability of immunosuppressant agents. The immunosuppressant used in the method and composition of the invention can be any agent which tends to attenuate the activity of the humoral or cellular immune systems. In particular, in one aspect the invention comprises a composition wherein the immunosuppressant is selected from the group consisting of cyclosporin A, FK506, prednisone, methylprednisolone, cyclophosphamide, thalidomide, azathioprine, and daclizumab, physalin B, physalin F, physalin G, seco-steroids purified from Physalis angulata L., 15-deoxyspergualin (DSG, 15-dos), MMF, rapamycin and its derivatives, CCI-779, FR 900520, FR 900523, NK86-1086, depsidomycin, kanglemycin-C, spergualin, prodigiosin25-c, cammunomicin, demethomycin, tetranactin, tranilast, stevastelins, myriocin, gliooxin, FR 651814, SDZ214-104, bredinin, WS9482, mycophenolic acid, mimoribine, misoprostol, OKT3, anti-IL-2 receptor antibodies, azasporine, leflunomide, mizoribine, azaspirane (SKF 105685), paclitaxel, altretamine, busulfan, chlorambucil, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea, and combinations thereof. In one more particular aspect, the immunosuppressant is cyclosporin A.

Furthermore, the therapeutic agent can be selected from the group consisting of a chemotherapeutic, a gene therapy vector, a growth factor, a contrast agent, an angiogenesis factor, a radionuclide, an anti-infection agent, an anti-tumor compound, a receptor-bound agent, a hormone, a steroid, a protein, a complexing agent, a polymer, a thrombin inhibitor, an antithrombogenic agent, a tissue plasminogen activator, a thrombolytic agent, a fibrinolytic agent, a vasospasm inhibitor, a calcium channel blocker, a nitrate, a nitric oxide promoter, a vasodilator, an antihypertensive agent, an antimicrobial agent, an antibiotic, a glycoprotein IIb/IIIa inhibitor, an inhibitor of surface glycoprotein receptors, an antiplatelet agent, an antimitotic, a microtubule inhibitor, a retinoid, an antisecretory agent, an actin inhibitor, a remodeling inhibitor, an antisense nucleotide, an agent for molecular genetic intervention, an antimetabolite, an antiproliferative agent, an anti-cancer agent, a dexamethasone derivative, an anti-inflammatory steroid, a non-steroidal antiinflammatory agent, an immunosuppressive agent, a PDGF antagonist, a growth hormone antagonist, a growth factor antibody, an anti-growth factor antibody, a growth factor antagonist, a dopamine agonist, a radiotherapeutic agent, an iodine-containing compound, a barium-containing compound, a heavy metal functioning as a radiopaque agent, a peptide, a protein, an enzyme, an extracellular matrix component, a cellular component, an angiotensin converting enzyme inhibitor, a 21-aminosteroid, a free radical scavenger, an iron chelator, an antioxidant, a sex hormone, an antipolymerases, an antiviral agent, an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents, gene therapy agents, a prodrug, a photodynamic therapy agent, and an agent for treating benign prostatic hyperplasia (BHP), a ¹⁴C-, ³H-, ¹³¹I-, ³²P- or ³⁶S-radiolabelled form or other radiolabelled form of any of the foregoing, and combinations thereof.

More particularly, the therapeutic agent can be selected from the group consisting of parathyroid hormone, heparin, human growth hormone, covalent heparin, hirudin, hirulog, argatroban, D-phenylalanyl-L-poly-L-arginyl chloromethyl ketone, urokinase, streptokinase, nitric oxide, triclopidine, aspirin, colchicine, dimethyl sulfoxide, cytochalasin, deoxyribonucleic acid, methotrexate, tamoxifen citrate, dexamethasone, dexamethasone sodium phosphate, dexamethasone acetate, cyclosporin, trapidal, angiopeptin, angiogenin, dopamine, ⁶⁰Co, ¹⁹²Ir, ³²P, ¹¹¹In, ⁹⁰Y, 99 mTc, pergolide mesylate, bromocriptine mesylate, gold, tantalum, platinum, tungsten, captopril, enalapril, ascorbic acid, α-tocopherol, superoxide dismutase, deferoxamine, estrogen, azidothymidine (AZT), acyclovir, famciclovir, rimantadine hydrochloride, ganciclovir sodium, 5-aminolevulinic acid, meta-tetrahydroxyphenylchlorin, hexadecafluoro zinc phthalocyanine, tetramethyl hematoporphyrin, and rhodamine 123, and combinations thereof.

The composition can further comprise one or more protease inhibitors. Any protease inhibitor can be used, including, but not limited to, a proteinase, peptidase, endopeptidase, or exopeptidase inhibitor. Certainly a cocktail of inhibitors can also be used, if appropriate. Alternatively, the protease inhibitors can be selected from the group consisting of bestatin, L-trans-3-carboxyoxiran-2-carbonyl-L-leucylagmatine, ethylenediaminetetraacetic acid (EDTA), phenylmethylsulfonylfluoride (PMSF), aprotinin, amyloid protein precursor (APP), amyloid beta precursor protein, α₁-proteinase inhibitor, collagen VI, bovine pancreatic trypsin inhibitor (BPTI), 4-(2-aminoethyl)-benzenesulfonyl fluoride (AEBSF), antipain, benzamidine, chymostatin, ε-aminocaproate, N-ethylmaleimide, leupeptin, pepstatin A, phosphoramidon, and combinations thereof. Novel protease inhibitors can also be used. Indeed, protease inhibitors can be specifically designed or selected to decrease the proteolysis of the tight junction agonist and/or the therapeutic agent.

Compositions of the invention can be formulated for intranasal delivery (e.g., can be intranasal dosage forms). Typically such compositions can be provided as pharmaceutical aerosols, e.g., solution aerosols. Those of skill in the art are aware of many different methods and devices for the formation of pharmaceutical aerosols, for example, those disclosed by Sciarra and Sciarra, Aerosols, in Remington: The Science and Practice of Pharmacy, 20th Ed., Chapter 50, Gennaro et al. Eds., Lippincott, Williams and Wilkins Publishing Co., (2000).

Typically, compositions comprising a tight junction agonist (e.g., peptide agonist) comprise a pharmaceutically effective amount of the agonist. The pharmaceutically effective amount of agonist (e.g., peptide agonist) employed may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation.

In one embodiment, the dosage forms are in the form of a solution aerosol (i.e., comprise droplets or particles). Typically, droplets or particles will be about 50 microns or less in diameter. Droplets or particles can be greater than about 5 microns in diameter. Droplets or particles for use in the compositions of the invention can have a diameter of from about 8 microns to about 50 microns, from about 8 microns to about 45 microns, from about 8 microns to about 40 microns, from about 8 microns to about 35 microns, from about 8 microns to about 30 microns, from about 8 microns to about 25 microns, from about 8 microns to about 20 microns, from about 20 microns to about 50 microns, from about 25 microns to about 50 microns, from about 30 microns to about 50 micron, from about 35 microns to about 50 microns, from about 40 microns to about 50 microns, from about 45 microns to about 50 microns, from about 20 microns to about 45 microns, from about 20 microns to about 40 microns, from about 20 micron to about 35 microns, from about 20 microns to about 30 microns, or from about 20 micron to about 25 microns. In some embodiments, particles and/or droplets for use in the invention may be about 20 microns, about 30 microns, about 40 microns, or about 50 microns in diameter.

Compositions of the invention may comprise one or tight junction agonist at a level of from about 0.000001 wt % to about 50 wt %, from about 0.000001 wt % to about 45 wt %, from about 0.000001 wt % to about 40 wt %, from about 0.000001 wt % to about 35 wt %, from about 0.000001 wt % to about 30 wt %, from about 0.000001 wt % to about 25 wt %, from about 0.000001 wt % to about 20 wt %, from about 0.000001 wt % to about 15 wt %, from about 0.000001 wt % to about 10 wt %, from about 0.000001 wt % to about 5 wt %, from about 0.000001 wt % to about 2.5 wt %, from about 0.000001 wt % to about 1 wt %, from about 0.000001 wt % to about 0.1 wt %, from about 0.000001 wt % to about 0.01 wt %, from about 0.000001 wt % to about 0.001 wt %, from about 0.000001 wt % to about 0.0001 wt %, from about 0.000001 wt % to about 0.00005 wt %, from about 0.0001 wt % to about 50 wt %, from about 0.0001 wt % to about 45 wt %, from about 0.0001 wt % to about 40 wt %, from about 0.0001 wt % to about 35 wt %, from about 0.0001 wt % to about 30 wt %, from about 0.0001 wt % to about 25 wt %, from about 0.0001 wt % to about 20 wt %, from about 0.0001 wt % to about 15 wt %, from about 0.0001 wt % to about 10 wt %, from about 0.0001 wt % to about 5 wt %, from about 0.0001 wt % to about 2.5 wt %, from about 0.0001 wt % to about 1 wt %, from about 0.0001 wt % to about 0.1 wt %, from about 0.0001 wt % to about 0.01 wt %, from about 0.0001 wt % to about 0.001 wt %, from about 0.0001 wt % to about 0.0005 wt %, from about 0.1 wt % to about 50 wt %, from about 0.1 wt % to about 45 wt %, from about 0.1 wt % to about 40 wt %, from about 0.1 wt % to about 35 wt %, from about 0.1 wt % to about 30 wt %, from about 0.1 wt % to about 25 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2.5 wt %, from about 0.1 wt % to about 1 wt %, from about 0.1 wt % to about 0.5 wt %, from about 0.1 wt % to about 0.2 wt %, from about 1 wt % to about 50 wt %, from about 1 wt % to about 45 wt %, from about 1 wt % to about 40 wt %, from about 1 wt % to about 35 wt %, from about 1 wt % to about 30 wt %, from about 1 wt % to about 25 wt %, from about 1 wt % to about 20 wt %, from about 1 wt % to about 15 wt %, from about 1 wt % to about 10 wt %, from about 1 wt % to about 5 wt %, from about 1 wt % to about 2.5 wt %, from about 5 wt % to about 50 wt %, from about 5 wt % to about 45 wt %, from about 5 wt % to about 40 wt %, from about 5 wt % to about 35 wt %, from about 5 wt % to about 30 wt %, from about 5 wt % to about 25 wt %, from about 5 wt % to about 20 wt %, from about 5 wt % to about 15 wt %, from about 5 wt % to about 10 wt %, from about 5 wt % to about 9 wt %, from about 5 wt % to about 8 wt %, from about 5 wt % to about 7 wt %, or from about 5 wt % to about 6 wt % of the total weight of the composition. Compositions of the invention may comprise one or more tight junction agonists at a level of about 0.00001 wt %, about 0.00005 wt %, about 0.0001 wt %, about 0.0005 wt %, about 0.001 wt %, about 0.005 wt %, about 0.01 wt %, about 0.05 wt %, about 0.1 wt %, about 0.5 wt %, about 1 wt %, about 5 wt %, about 10 wt %, about 15 wt %, about 20 wt %, about 25 wt %, about 30 wt %, about 35 wt %, about 40 wt %, about 45 wt %, or about 50 wt % based on the total weight of the composition.

Compositions of the invention may comprise one or more therapeutic agents at a concentration sufficient to cause the desired biological response (e.g., at a pharmaceutically effective concentration). Compositions of the invention may comprise one or therapeutic agents at a level of from about 0.1 wt % to about 50 wt %, from about 0.1 wt % to about 45 wt %, from about 0.1 wt % to about 40 wt %, from about 0.1 wt % to about 35 wt %, from about 0.1 wt % to about 30 wt %, from about 0.1 wt % to about 25 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2.5 wt %, from about 0.1 wt % to about 1 wt %, from about 0.1 wt % to about 0.5 wt %, from about 0.1 wt % to about 0.2 wt %, from about 1 wt % to about 50 wt %, from about 1 wt % to about 45 wt %, from about 1 wt % to about 40 wt %, from about 1 wt % to about 35 wt %, from about 1 wt % to about 30 wt %, from about 1 wt % to about 25 wt %, from about 1 wt % to about 20 wt %, from about 1 wt % to about 15 wt %, from about 1 wt % to about 10 wt %, from about 1 wt % to about 5 wt %, from about 1 wt % to about 2.5 wt %, from about 5 wt % to about 50 wt %, from about 5 wt % to about 45 wt %, from about 5 wt % to about 40 wt %, from about 5 wt % to about 35 wt %, from about 5 wt % to about 30 wt %, from about 5 wt % to about 25 wt %, from about 5 wt % to about 20 wt %, from about 5 wt % to about 15 wt %, from about 5 wt % to about 10 wt %, from about 5 wt % to about 9 wt %, from about 5 wt % to about 8 wt %, from about 5 wt % to about 7 wt %, or from about 5 wt % to about 6 wt % of the total weight of the composition. Compositions of the invention may comprise one or more therapeutic agents at a level of about 0.1 wt %, about 1 wt %, about 5 wt %, about 10 wt %, about 15 wt %, about 20 wt %, about 25 wt %, about 30 wt %, about 35 wt %, about 40 wt %, about 45 wt %, or about 50 wt % based on the total weight of the composition.

Compositions of the invention may comprise one or pharmaceutically acceptable excipients at a level of from about 0.1 wt % to about 50 wt %, from about 0.1 wt % to about 45 wt %, from about 0.1 wt % to about 40 wt %, from about 0.1 wt % to about 35 wt %, from about 0.1 wt % to about 30 wt %, from about 0.1 wt % to about 25 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2.5 wt %, from about 0.1 wt % to about 1 wt %, from about 0.1 wt % to about 0.5 wt %, from about 0.1 wt % to about 0.2 wt %, from about 1 wt % to about 50 wt %, from about 1 wt % to about 45 wt %, from about 1 wt % to about 40 wt %, from about 1 wt % to about 35 wt %, from about 1 wt % to about 30 wt %, from about 1 wt % to about 25 wt %, from about 1 wt % to about 20 wt %, from about 1 wt % to about 15 wt %, from about 1 wt % to about 10 wt %, from about 1 wt % to about 5 wt %, from about 1 wt % to about 2.5 wt %, from about 5 wt % to about 50 wt %, from about 5 wt % to about 45 wt %, from about 5 wt % to about 40 wt %, from about 5 wt % to about 35 wt %, from about 5 wt % to about 30 wt %, from about 5 wt % to about 25 wt %, from about 5 wt % to about 20 wt %, from about 5 wt % to about 15 wt %, from about 5 wt % to about 10 wt %, from about 5 wt % to about 9 wt %, from about 5 wt % to about 8 wt %, from about 5 wt % to about 7 wt %, or from about 5 wt % to about 6 wt % of the total weight of the composition. Compositions of the invention may comprise one or more pharmaceutically acceptable excipients at a level of about 0.1 wt %, about 1 wt %, about 5 wt %, about 10 wt %, about 15 wt %, about 20 wt %, about 25 wt %, about 30 wt %, about 35 wt %, about 40 wt %, about 45 wt %, or about 50 wt % based on the total weight of the composition. Suitable excipients include, but are not limited to, salts, buffers polymers and the like.

A composition according to the present invention may be pre-mixed prior to administration, or can be formed in vivo when two or more components (e.g., a tight junction agonist and a therapeutic agent) are administered within 24 hours of each other. When administered separately, the components may be administered in either order (e.g. tight junction agonist first followed by therapeutic agent or therapeutic agent first followed by tight junction agonist). The components can be administered within a time span of about 12 hours, about 8 hours, about 4 hours, about 2 hours, about 1 hour, about 0.5 hour, about 0.25 hour, about 0.1 hour, about 1 minute, about 0.5 minute, or about 0.1 minute.

Methods of Use

The pharmaceutical compositions of the invention can be used for treating, ameliorating, and/or preventing a disease. Any disease may be treated using the compositions of the invention by selection of an appropriate therapeutic and/or immunogenic agent. In one embodiment, the present invention provides a method of treating diabetes by administering a composition comprising one or more tight junction agonist and one or more insulin and/or derivative thereof. In another embodiment, the invention provides a method of suppressing an excessive or undesirable immune response in a subject (e.g., a mammal such as a human) by administering a composition comprising a tight junction agonist and an immune-suppressive drug, for example, cyclosporin A.

Examples of diseases that can be treated using the compositions of the invention include, but are not limited to, cancer, autoimmune diseases, vascular disease, bacterial infections, gastritis, gastric cancer, collagnenous colitis, inflammatory bowel disease, osteoporosis, systemic lupus erythematosus, food allergy, asthma, and irritable bowel syndrome. For example, to treat cancer of the colon or rectal area, a composition comprising a therapeutically effective amount of Erbitux (Cetuximab) and an absorption enhancing amount of one or more tight junction agonists may be administered to the nose of a subject (e.g., a mammal such as a human) in need thereof, to treat breast cancer, a composition comprising a therapeutically effective amount of Herceptin (Trastuzumab) and an absorption enhancing amount of one or more tight junction agonists may be administered to the nose of a subject (e.g., a mammal such as a human) in need thereof, and to treat various types of cancer, a composition comprising a therapeutically effective amount of Avastin (Bevacizumab) and an absorption enhancing amount of one or more tight junction agonist may be administered to the nose of a subject (e.g., a mammal such as a human) in need thereof. Further examples include treatment of osteoporosis using a composition comprising one or more tight junction agonists and a therapeutically effective amount of Fosamax (Alendronate) administered to the lung of a subject in need thereof, treatment of transplant rejection using a composition comprising one or more tight junction agonists and a therapeutically effective amount of Cyclosporin A administered to the lung of a subject in need thereof, treatment of anemia using a composition comprising one or more tight junction agonists and a therapeutically effective amount of erythropoietin administered to the lung of a subject in need thereof, and treatment of hemophilia using a composition comprising one or more tight junction agonists and a therapeutically effective amount of Factor VIII administered to the lung of a subject in need thereof.

The following examples are provided for illustrative purposes only, and are in no way intended to limit the scope of the present invention.

### EXAMPLES

[hu 3H]-Cyclosporin A (CsA; 8Ci/mM, 1 mCi/ml) was purchased from Amersham Radiochemicals (Piscataway, N.J.). [¹⁴C]-Mannitol (46.6 mCi/mM, 60 μCi/ml) was purchased from Sigma Chemical Co. (St. Louis, Mo.). All chemicals were of analytical grade. All surgical supplies were purchased from World Precision Instruments (Sarasota, Fla.). Polyethylene 50 (PE50) tubing was obtained from Clay Adams (Parsippany, N.J.). Universol Scintillation counting cocktail was purchased from ICN (Cost Mesa, Calif.). The Caco-2 cell line was obtained from American Tissue Culture Collection (ATCC; Rockville, Md.). Caco-2 cell culture supplies (Dulbecco's modified Eagle medium, phosphate buffer saline (PBS), non essential amino acids, fetal bovine serum, L-glutamate, trypsin (0.25%)-EDTA (1 mM), and Penicillin G-streptomycin sulfate antibiotic mixture) were purchased from Gibco Laboratories (Lenexa, Kans.). Transwell clusters, 12-well (3 μm pores, surface area 1 cm²) were purchased from Corning Costar (Cambridge, Mass.).

Caco-2 cells, a human colon adenocarcinoma cell line, were grown as monolayers for 21 days in Dulbecco's Modified Eagle's medium (1X) containing 10% fetal bovine serum, 1% non-essential amino acid solution, 1% penicillin-streptomycin and 2% glutamine at 37° C. in an atmosphere of 5% CO₂ and 90% relative humidity. Caco-2 cells from passage numbers of 51 to 52 were seeded on permeable polycarbonate inserts (1 cm², 0.4 μm pore size) in 12 Transwell plates at a density of 80,000 cells/cm². The inserts were fed with media every other day until they were used for experiments 21 days after the initial seeding. The integrity of the cell monolayers was evaluated by measuring the transepithelial electrical resistance (TEER) values before the study using a Millicell®-ERS meter (Millipore Corp., Bedford, Mass.) with chopstick electrodes. The transport of [¹⁴C]-mannitol was also performed prior to the transport studies. The cell monolayers were considered to be tight when the apparent permeability coefficients (Pₐₚₚ) value of [¹⁴C]-mannitol was <1×10⁻⁶ cm/s. The cell monolayers were washed twice with PBS prior to the transport experiments. After the wash, the plates were incubated for 30 min at 37° C., and the integrity of the cell monolayers was evaluated by measurement of TEER. The cell inserts were used in transport experiments when the TEER values reached >300 Ωcm².

To measure the apical to basolateral transport of CsA, 0.5 ml of each CsA treatment, i.e., (1) the PBS solution of CsA, (2) the PBS solution of CsA/PI, (3) the PBS solution of CsA/PI/BC, (4) the PBS solution of CsA/AT1002, (5) the PBS solution of CsA/PI/AT1002, and (6) the PBS solution of CsA/PI/BC/AT1002 (CsA 0.5 μCi/ml, PI (bestatin 15 mM and E64 5 mM), BC 0.005 w/v % (benzalkonium chloride), and AT1002 5 mM, respectively) was added to the apical side, and 1.5 ml of PBS was added to the basolateral side of the insert. The insert was moved to a well containing fresh PBS every 10 min for 40 min. Samples were collected from the basolateral side of each well, and the radioactivity of CsA transported was measured by Beckman Coulter LS 6500 multi-purpose Scintillation counter.

Mice were housed in cages and allowed to acclimate at least two days after arrival. Mice were fed chow and water ad libitum and maintained on a 12-h light: 12-h dark cycle. The protocol for the animal studies was approved by the School of Pharmacy, University of Maryland IACUC.

Blood samples (250 μl) were drawn into heparinized syringes at 10 or 15 min into polypropylene tubes, centrifuged (13,000 rpm for 10 min) immediately and plasma was obtained. Scintillation cocktail was added and samples were analyzed for radioactivity by Beckman Coulter LS 6500 multi-purpose Scintillation counter.

Pₐₚₚ was calculated according to the following equation:

$${P}_{app} = \frac{d Q}{d t} \frac{Vr}{A \cdot {D}_{0}}$$

Where dQ/dt is equal to the linear appearance rate of mass in the receiver solution, A is the cross sectional area (1 cm²), Do is equal to the initial amount in the donor compartment, Vr is equal to the volume of the receiver compartment (1.5 ml).

The percent enhancement ratio, ER(%), for the Pₐₚₚ was calculated from the formula,

$$ER ( \% ) = \frac{{P}_{app} {\cdot }_{( treatment )}}{{P}_{app} {\cdot }_{( control )}} \times 100$$

All data are expressed as the mean and standard error of the mean of the values (mean±SEM). The statistical significance of differences between treatments and/or controls was evaluated using the Student's t-test and Analysis of variance followed by Dunnett's post hoc test (SPSS for Windows versions 12.0., SPSS Inc., Chicago, Ill.) (p<0.05 or p<0.01).

### Example 1

Caco-2 transport studies of CsA with AT1002

Many therapeutically active agents experience low bioavailability after oral administration due to poor absorption or susceptibility to first pass metabolism. Transient opening of TJ to improve paracellular drug transport and increase oral absorption would be beneficial to the therapeutic effect. Absorption enhancers are capable of modulation of TJ to improve the transport or absorption of low BA drugs. However, some absorption enhancers cause serious damage to the epithelial integrity, morphology and function (14).

Cyclosporin A(CsA), a major immunosuppressive drug, exhibits a low therapeutic index and a mean BA of ˜20% (15). As such, CsA transport provides a useful model for evaluating methods and compositions for enhancing BA by enhancing transport across epithelial cell layers. Table 1 summarizes the permeability coefficients (Pₐₚₚ) associated with the various transport studies performed with AT1002 and CsA. The apparent permeability coefficients were determined across Caco-2 cell monolayers (Pₐₚₚ) of mannitol, CsA, and CsA with added agents: mannitol 0.5 μCi/ml, CsA 0.5 μCi/ml, PI (bestatin 15 mM and E-64 5 mM), BC 0.005 w/v %, and/or AT1002 5mM. Data are presented as mean±SEM (n=3).

TABLE I

| | Pₐₚₚ (×10⁻⁶ cm/sec) | ER (%) |
|---|---|---|
| Mannitol | 0.69 ± 0.06 | — |
| CsA | 1.28 ± 0.10 | — |
| CsA/AT1002 | 1.54 ± 0.13 | 120 |
| CsA/PI | 1.59 ± 0.07 | — |
| CsA/PI/AT1002 | 1.76 ± 0.05 | 111 |
| CsA/PI/BC | 1.60 ± 0.03 | — |
| CsA/PI/BC/AT1002 | 1.52 ± 0.06 | 95 |

The mean Pₐₚₚ determined for CsA were 1.28±0.10, 1.54±0.13, 1.59±0.07, 1.76±0.05, 1.60±0.03, and 1.52±0.06 (×10⁻⁶ cm/sec, mean±SEM, n=3), for the following treatments CsA, CsA/AT1002, CsA/PI, CsA/PI/AT1002, CsA/PI/BC, and CsA/PI/BC/AT1002, respectively. The fold increases of CsA across Caco-2 cell monolayers were 120%, 111%, and 95% after the following treatments CsA/AT1002, CsA/PI/AT1002, and CsA/PI/BC/AT1002 treatment compared to each of the following controls, CsA, CsA/PI, and CsA/PI/BC, respectively. Thus, although AT1002 and AT1002 in the presence of protease inhibitors increased CsA transport, the increases in Pₐₚₚ and the fold-increases over controls were not statistically significant. Mannitol permeability was found to be 6.86±0.57×10⁻⁷ cm/sec suggesting integrity of the tight junctions in the Caco-2 cells.

### Example 2

Intranasal administration of AZT with AT1002 to FVB mice.

In a volume of 20 μl, 120 μCi/kg of [³H]-AZT was administered to FVB mice of 20-25 g each. Other FVB mice received the same amount of AZT administered in the presence of AT1002(2 mg/kg). Blood samples were taken at 10 or 15 minutes.

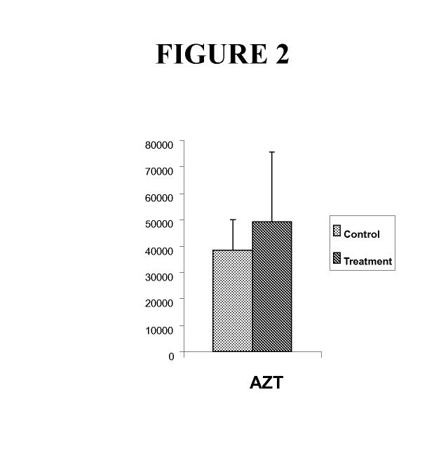

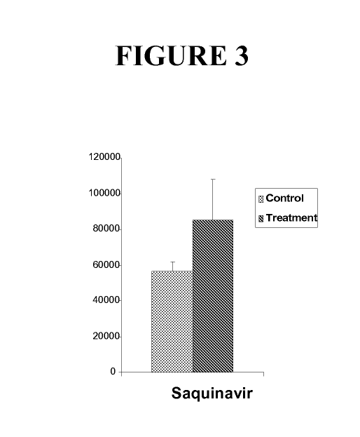

FIG. 2 illustrates the results. Co-administration of AT1002 significantly (p<0.05) increased levels of AZT observation blood plasma. See FIG. 2. Indeed, the co-administration of AT1002 increased the AZT levels in plasma by about 33%.

[hu 3H]-Saquinavir (120 μCi/kg) was administered in a 20 μl volume intranasally to FVB mice in the absence or presence of AT1002 (5 mg/kg). AT1002 increased the levels of saquinavir in the plasma by about 51%. See FIG. 3.

### Example 3

AT1002 is a six-mer synthetic peptide, H-FCIGRL-OH, that retains the Delta G and ZOT biological activity of reversibly opening tight junctions and increases the paracellular transport of drugs. The objective of this study was to evaluate the possible use of AT1002 in enhancing the nasal availability of macromolecules using large paracelluar markers as model agents.

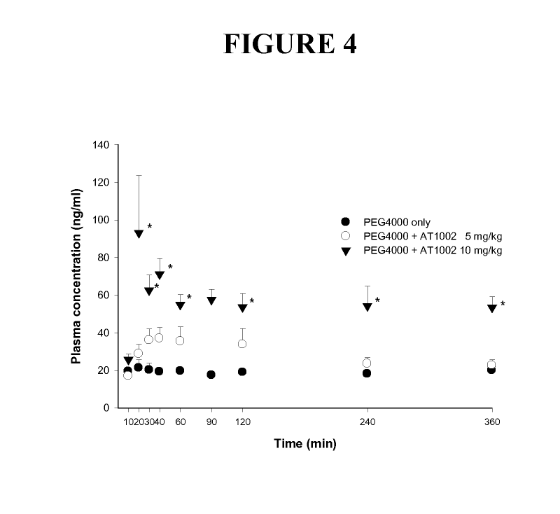

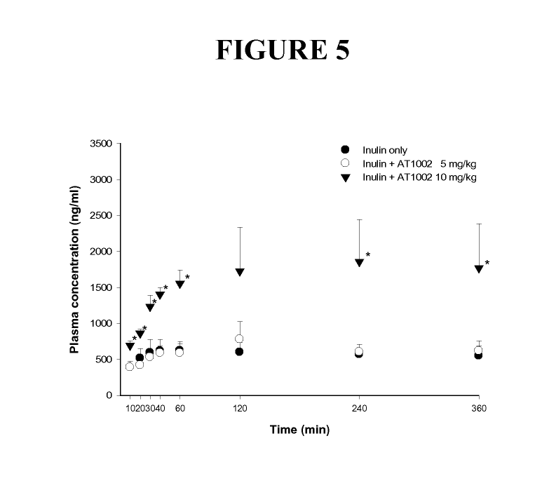

Male Sprague-Dawley rats cannulated in the jugular vein were randomly assigned to receive radiolabelled paracellular markers, [¹⁴C] PEG4000 or [¹⁴C] inulin, with/without AT1002, for each intranasal study. As shown in FIG. 4, the plasma concentration of PEG4000 with AT1002 (10 mg/kg) was significantly higher than that from PEG4000 control over 360 min following intranasal administration. The AUC₀₋₃₆₀ₘᵢₙ and Cₘₐₓ from the PEG4000/AT1002 (10 mg/kg) treatment were statistically (p<0.05) increased to 235% and 357%, of control, respectively. As shown in FIG. 5, when inulin was administered with AT1002 (10 mg/kg), the plasma concentration was significantly higher (p<0.05) than control over 360 min, and increases (p<0.05) of 292% and 315% for AUC₀₋₃₆₀ₘᵢₙ and Cₘₐₓ over control were observed, respectively. AT1002 significantly increased the nasal absorption of molecular weight markers, PEG4000 and inulin. This study suggests that AT1002 may be used to enhance the systemic availability of macromolecules when administered concurrently.

### Example 4

Salmon calcitonin (sCT) is a clinically useful drug in the treatment of a variety of bone diseases. AT1002, a six-mer peptide, was isolated as a tight junction modulating peptide from Zonula Occludens Toxin and delta G. The purpose of this study was to investigate the feasibility of sCT enhancement by intra-nasal delivery with AT1002, permeation enhancer to modulate the tight junction.

Jugular cannulated Sprague-Dawley rats randomly received formulations of salmon calcitonin. The formulations administered intra-nasally to rats were dextrose solution of sCT (15 mg/kg) with or without various doses of AT1002 (2, 5, and 10 mg/kg). Doses were then slowly administered intra-nasally with a volume dose of 400 μl kg rat. Blood samples were drawn via the jugular cannula at each time point, and were deproteinized by the addition of acetonitrile. The concentration of sCT in the plasma was determined by LC-MS.

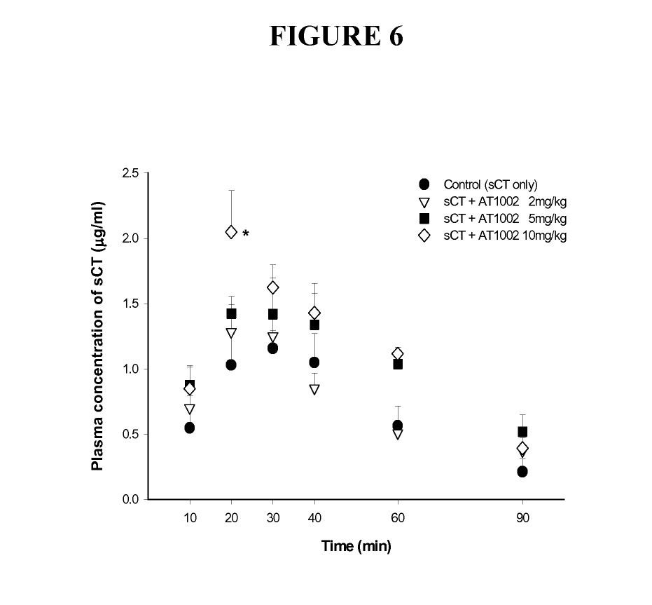

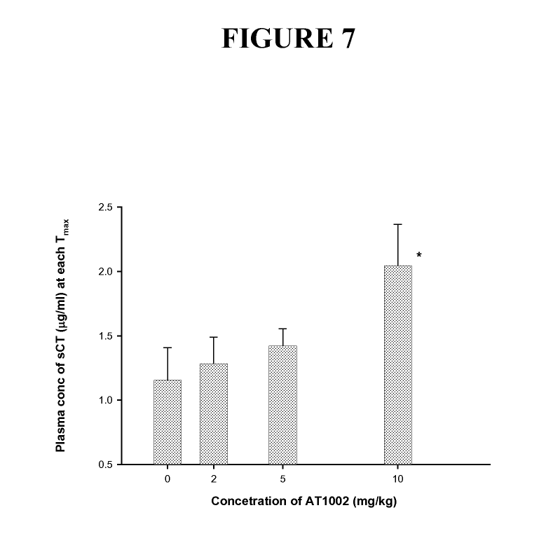

As shown in FIG. 6, the plasma concentration of sCT was increased by 1.99-fold statistically (p<0.05) and significantly higher than that from sCT control at 20 min, when sCT was administered with 10 mg/kg of AT1002. The pharmacokinetic profile displayed statistical (p<0.05) increases in the rate and extent of absorption for a period of 90 min with 1.66-fold increase in AUC₀₋₉₀ₘᵢₙ (99.24±5.41 min μg/ml) and 1.67-fold increase in Cₘₐₓ(2.05±0.32 μg/ml) to those of the sCT control. FIG. 7 shows the plasma concentrations at Tmax for the various treatments.

TABLE 2
Mean ± SEM absorption parameters for sCT (15 mg/kg)
after intra-nasal administration to jugular vein cannulated
Sprague-Dawley rats (n = 3-5) alone and/or with AT1002.
ER was calculated to sCT only (control).

| | AUC_{0-90 min} | | Tₘₐₓ |
|---|---|---|---|
| Treatments | (min μg/ml) | Cₘₐₓ (μg/ml) | (min) |
| sCT only | 59.82 ± 13.53 | 1.23 ± 0.25 | 32.50 ± 2.50 |
| (control) | | | |
| sCT + AT1002 | 63.38 ± 4.97 | 1.38 ± 0.11 | 23.33 ± 3.33 |
| (2 mg/kg) | (1.06) | (1.13) | (0.72) |
| sCT + AT1002 | 83.00 ± 15.14* | 1.69 ± 0.27* | 26.00 ± 4.00 |
| (5 mg/kg) | (1.39) | (1.38) | (0.80) |
| sCT + AT1002 | 99.24 ± 5.41* | 2.05 ± 0.32* | 22.50 ± 2.50 |
| (10 mg/kg) | (1.66) | (1.67) | (0.69) |

*p < 0.05, significantly different from control (sCT control)

The in vivo study indicated the potential of AT1002 as effective permeation enhancer of peptides like sCT after intra-nasal administration. This addition of information about AT1002 might be useful of the drug delivery of peptides and low bioavailable therapeutic agents.

While the invention has been described in detail, and with reference to specific embodiments thereof, it will be apparent to one of ordinary skill in the art that various changes and modifications can be made therein without departing from the spirit and scope thereof and such changes and modifications may be practiced within the scope of the appended claims. All patents and publications herein are incorporated by reference to the same extent as if each individual publication was specifically and individually indicated to be incorporated by reference in their entirety.

### REFERENCES

1. B. J. Aungst. Intestinal Permeation enhancers. J Pharm Sci. 89(4):429-442 (2000).
2. A. Fasano, B. Baudry D. W. Pumplin, S. S. Wasserman, B. D. Tall, J. M. Ketley, and J. B. Kaper. Vibrio cholerae produces a second enterotoxin, which affects intestinal tight junctions. Proc Natl Acad Sci USA. 88(12):5242-5246 (1991).
3. A. Fasano, C. Fiorentini, G. Donelli, S. Uzzau, J. B. Kaper, K. Margaretten, X. Ding, S. Guandalini, L. Comstock, and S. E. Goldblum. Zonula occludens toxin modulates tight junctions through protein kinase C-dependent actin reorganization, in vitro. J Clin Invest. 96(2):710-720 (1995).
4. A. Fasano and S. Uzzau. Modulation of intestinal tight junctions by Zonula occludens toxin permits enteral administration of insulin and other macromolecules in an animal model. J Clin Invest. 99(6):1158-1164 (1997).
5. A. Fasano, S. Uzzau, C. Fiore, and K. Margaretten. The enterotoxic effect of zonula occludens toxin on rabbit small intestine involves the paracellular pathway. Gastroenterology. 112(3):839-846 (1997).
6. D. S. Cox, H. Gao, S. Raje, K. R. Scott, and N. D. Eddington. Enhancing the permeation of marker compounds and enaminone anticonvulsants across Caco-2 monolayers by modulating tight junctions using zonula occludens toxin. Eur J Pharm Biopharm. 52(2):145-150 (2001).
7. D. S. Cox, S. Raje, H. Gao, N. N. Salama, and N. D. Eddington. Enhanced permeability of molecular weight markers and poorly bioavailable compounds across Caco-2 cell monolayers using the absorption enhancer, zonula occludens toxin. Pharm Res. 19(11):1680-1688 (2002).
8. M. Di Pierro, R. Lu, S. Uzzau, W. Wang, K. Margaretten, C. Pazzani, F. Maimone, and A. Fasano. Zonula occludens toxin structure-function analysis. Identification of the fragment biologically active on tight junctions and of the zonulin receptor binding domain. J Biol Chem. 276(22):19160-19165 (2001).
9. N. Cenac, A. C. Chin, R. Garcia-Villar, C. Salvador-Cartier, L. Ferrier, N. Vergnolle, A. G. Buret, J. Fioramonti, and L. Bueno. PAR2 activation alters colonic paracellular permeability in mice via IFN-gamma-dependent and -independent pathways. J. Physiol. 558(Pt 3):913-925 (2004).
10. N. N. Salama, A. Fasano, M. Thakar, and N. D. Eddington. The effect of delta G on the transport and oral absorption of macromolecules. J Pharm Sci. 93(5):1310-1319 (2004). 11. N. N. Salama, A. Fasano, R. Lu, and N. D. Eddington. Effect of the biologically active fragment of zonula occludens toxin, delta G, on the intestinal paracellular transport and oral absorption of mannitol. Int J Pharm. 251(1-2):113-121 (2003).
12. N. N. Salama, A. Fasano, M. Thakar, and N. D. Eddington. The impact of DeltaG on the oral bioavailability of low bioavailable therapeutic agents. J Pharmacol Exp Ther. 312(1):199-205 (2005).
13. C. S. Karyekar, A. Fasano, S. Raje, R. Lu, T. C. Dowling, and N. D. Eddington N. Dak. Zonula occludens toxin increases the permeability of molecular weight markers and chemotherapeutic agents across the bovine brain microvessel endothelial cells. J Pharm Sci. 92(2):414-423 (2003).
14. M. Thanou, B. I. Florea, M. W. Langemeyer, J. C. Verhoef, H. E. Junginger. N-trimethylated chitosan chloride (TMC) improves the intestinal permeation of the peptide drug buserelin in vitro (Caco-2 cells) and in vivo (rats). Pharm Res. 17(1):27-31 (2000).

15. Y. Ogino, E. Kobayashi, and A. Fujimura. Comparison of cyclosporin A and tacrolimus concentrations in whole blood between jejunal and ileal transplanted rats. J Pharm Pharmacol. 51(7):811-815 (1999).

What is claimed is:

1. A therapeutic composition comprising
a therapeutically effective amount of one or more therapeutic agents and a nasal mucosa absorption enhancing amount of one or more tight junction agonists.

2. The composition of claim 1 wherein at least one of the one or more tight junction agonists comprises a peptide.

3. The composition of claim 2 wherein the peptide comprises from about 6 to about 10 amino acid residues.

4. The composition of claim 2 wherein the peptide comprises a sequence selected from the group consisting of SEQ ID NOs: 1-22.

5. The composition of claim 2 wherein at least one of the one or more tight junctions is a peptide comprising the sequence FCIGRL.

6. The composition of claim 1 wherein at least one therapeutic agent is selected from the group consisting of an antibiotic, an anti-inflammatory, an analgesic, an immunosuppressant, and a peptide hormone.

7. The composition of claim 6 wherein the immunosuppressant is selected from the group consisting of cyclosporin A, FK506, prednisone, methylprednisolone, cyclophosphamide, thalidomide, azathioprine, and daclizumab, physalin B, physalin F, physalin G, seco-steroids purified from Physalis angulata L., 15-deoxyspergualin, MMF, rapamycin and its derivatives, CCI-779, FR 900520, FR 900523, NK86-1086, depsidomycin, kanglemycin-C, spergualin, prodigiosin25-c, cammunomicin, demethomycin, tetranactin, tranilast, stevastelins, myriocin, gliotoxin, FR 651814, SDZ214-104, bredinin, WS9482, mycophenolic acid, mimoribine, misoprostol, OKT3, anti-IL-2 receptor antibodies, azasporine, leflunomide, mizoribine, azaspirane, paclitaxel, altretamine, busulfan, chlorambucil, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea, and combinations thereof.

8. The composition of claim 6 wherein the immunosuppressant is cyclosporin A.

9. The composition of claim 6 wherein the peptide hormone is insulin.

10. The composition of claim 1 wherein at least one of the one or more therapeutic agents is selected from the group consisting of a small molecule, a peptide, a protein, a lipid, a carbohydrate, and combinations thereof.

11. The composition of claim 1 wherein at least one of the one or more therapeutic agents is selected from the group consisting of a chemotherapeutic, a gene therapy vector, a growth factor, a contrast agent, an angiogenesis factor, a radionuclide, an anti-infection agent, an anti-tumor compound, a receptor-bound agent, a hormone, a steroid, a protein, a complexing agent, a polymer, a thrombin inhibitor, an antithrombogenic agent, a tissue plasminogen activator, a thrombolytic agent, a fibrinolytic agent, a vasospasm inhibitor, a calcium channel blocker, a nitrate, a nitric oxide promoter, a vasodilator, an antihypertensive agent, an antimicrobial agent, an antibiotic, a glycoprotein IIb/IIIa inhibitor, an inhibitor of surface glycoprotein receptors, an antiplatelet agent, an antimitotic, a microtubule inhibitor, a retinoid, an antisecretory agent, an actin inhibitor, a remodeling inhibitor, an antisense nucleotide, an agent for molecular genetic intervention, an antimetabolite, an antiproliferative agent, an anti-cancer agent, a dexamethasone derivative, an anti-inflammatory steroid, a non-steroidal antiinflammatory agent, an immunosuppressive agent, a PDGF antagonist, a growth hormone antagonist, a growth factor antibody, an anti-growth factor antibody, a growth factor antagonist, a dopamine agonist, a radiotherapeutic agent, an iodine-containing compound, a barium-containing compound, a heavy metal functioning as a radiopaque agent, a peptide, a protein, an enzyme, an extracellular matrix component, a cellular component, an angiotensin converting enzyme inhibitor, a 21-aminosteroid, a free radical scavenger, an iron chelator, an antioxidant, a sex hormone, an antipolymerases, an antiviral agent, an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents, gene therapy agents, a prodrug, a photodynamic therapy agent, and an agent for treating benign prostatic hyperplasia (BHP), a ¹⁴C-, ³H-, ¹³¹I-, ³²P- or ³⁵S-radiolabelled form or other radiolabelled form of any of the foregoing, and combinations thereof.

12. The composition of claim 1 wherein at least one of the one or more therapeutic agents is selected from the group consisting of parathyroid hormone, heparin, human growth hormone, covalent heparin, hirudin, hirulog, argatroban, D-phenylalanyl-L-poly-L-arginyl chloromethyl ketone, urokinase, streptokinase, nitric oxide, triclopidine, aspirin, colchicine, dimethyl sulfoxide, cytochalasin, deoxyribonucleic acid, methotrexate, tamoxifen citrate, dexamethasone, dexamethasone sodium phosphate, dexamethasone acetate, cyclosporin, trapidal, angiopeptin, angiogenin, dopamine, 60Co, 192Ir, 32P, 111In, 90Y, 99 mTc, pergolide mesylate, bromocriptine mesylate, gold, tantalum, platinum, tungsten, captopril, enalapril, ascorbic acid, α-tocopherol, superoxide dismutase, deferoxamine, estrogen, AZT, acyclovir, famciclovir, rimantadine hydrochloride, ganciclovir sodium, 5-aminolevulinic acid, meta-tetrahydroxyphenylchlorin, hexadecafluoro zinc phthalocyanine, tetramethyl hematoporphyrin, and rhodamine 123, and combinations thereof.

13. The composition of claim 1 wherein the composition is in aqueous solution.

14. The composition of claim 1 further comprising one or more pharmaceutically acceptable excipients.

15. The composition of claim 14 wherein at least one of the one or more tight junctions is a peptide comprising the sequence FCIGRL and the composition further comprises one or more therapeutic agents selected from the group consisting of a small molecule, a peptide, a protein, a protease inhibitor, a lipid, and a carbohydrate, and combinations thereof.

16. A method of treating a subject comprising:
intranasally administering to the subject a composition comprising one or more therapeutic agents and a nasal mucosa absorption enhancing amount of one or more tight junction agonists.

17. The method of claim 16 wherein the subject is a mammal.

18. The method of claim 16 wherein the subject is a human.

19. The method of claim 16 wherein at least one of the one or more tight junctions comprises a peptide.

20. The method of claim 19 wherein the peptide comprises from about 6 to about 10 amino acid residues.

21. The method of claim 19 wherein the peptide comprises a sequence selected from the group consisting of SEQ ID NOs: 1-22.

22. The method of claim 16 wherein at least one of the one or more tight junctions is a peptide comprising the sequence FCIGRL.

23. The method of claim 16 wherein at least one of the one or more therapeutic agents is selected from the group consisting of an antibiotic, an anti-inflammatory, an analgesic, an immunosuppressant, and a peptide hormone.

24. The method of claim 23 wherein the immunosuppressant is selected from the group consisting of cyclosporin A, FK506, prednisone, methylprednisolone, cyclophosphamide, thalidomide, azathioprine, and daclizumab, physalin B, physalin F, physalin G, seco-steroids purified from Physalis angulata L., 15-deoxyspergualin, MMF, rapamycin and its derivatives, CCI-779, FR 900520, FR 900523, NK86-1086, depsidomycin, kanglemycin-C, spergualin, prodigiosin25-c, cammunomicin, demethomycin, tetranactin, tranilast, stevastelins, myriocin, gliooxin, FR 651814, SDZ214-104, bredinin, WS9482, mycophenolic acid, mimoribine, misoprostol, OKT3, anti-IL-2 receptor antibodies, azasporine, leflunomide, mizoribine, azaspirane, paclitaxel, altretamine, busulfan, chlorambucil, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea, and combinations thereof.

25. The method of claim 23 wherein the immunosuppressant is cyclosporin A.

26. The method of claim 23 wherein the peptide hormone is insulin.

27. The method of claim 16 wherein at least one of the one or more therapeutic agents is selected from the group consisting of a small molecule, a peptide, a protein, a lipid, a carbohydrate, and combinations thereof.

28. The method of claim 16 wherein the composition is an aqueous solution.

29. The method of claim 16 wherein the composition further comprises one or more pharmaceutically acceptable excipients.

30. The method of claim 16 wherein at least one of the one or more tight junction agonists is a peptide comprising the sequence FCIGRL and the composition further comprises at least one protease inhibitor and one or more therapeutic agents selected from the group consisting of a small molecule, a peptide, a protein, a lipid, and a carbohydrate, and combinations thereof.

31. A method of treating diabetes in a subject in need thereof, comprising:
intranasally administering to the subject a composition comprising insulin, a derivative of insulin, or a combination thereof, and a nasal mucosa absorption enhancing amount of one or more tight junction agonists.

32. The method of claim 31 wherein the subject is a mammal.

33. The method of claim 31 wherein the subject is a human.

34. The method of claim 31 wherein at least one of the one or more tight junction agonists comprises a peptide.

35. The method of claim 34 wherein the peptide comprises from about 6 to about 10 amino acid residues.

36. The method of claim 34 wherein the peptide is selected from the group consisting of SEQ ID NOs: 1-22.

37. The method of claim 31 wherein at least one of the one or more tight junction agonists is a peptide comprising the sequence FCIGRL.

38. The method of claim 31 wherein the composition is in aqueous solution.

39. The method of claim 31 wherein the composition further comprises one or more pharmaceutically acceptable excipients.
